(19) United States
(12) Patent Application Publication
(10) Pub. No.: US 2026/0007719 A1
(43) Pub. Date: Jan. 8, 2026

(54) COMPOSITION FOR INTERMITTENT DOSING OF CALCINEURIN INHIBITORS

(71) Applicant: Trevor Klee, Boston, MA (US)

(72) Inventor: Trevor Klee, Boston, MA (US)

(21) Appl. No.: 18/881,766

(22) PCT Filed: Jul. 7, 2023
(86) PCT No.: PCT/US23/27060

§ 371 Date: Jan. 7, 2025

Related U.S. Application Data
(60) Provisional application No. 63/359,350, filed on Jul. 8, 2022.
(60) Provisional application No. 63/456,017, filed on Mar. 31, 2023.

(51) Int. Cl. A61K 38/13; A61K 31/427; A61K 31/436; A61K 45/06

(52) U.S. Cl. CPC A61K 38/13; A61K 31/427; A61K 31/436; A61K 45/06

(57) ABSTRACT

Provided herein are compositions and dosing regimens of a calcineurin inhibitor and a cytochrome p450 inhibitor.

### CROSS-REFERENCE TO RELATED APPLICATIONS

The present application claims priority to U.S. Provisional Application No. 63/359,350, filed Jul. 8, 2022, and U.S. Provisional Application No. 63/456,017, filed Mar. 31, 2023, each of which is incorporated by reference in its entirety.

### FIELD OF TECHNOLOGY

The present disclosure further provides methods of administering a calcineurin inhibitor (e.g. cyclosporine) in combination with a cytochrome p450 enzyme inhibitor (e.g. ritonavir) in a intermittent dosing (e.g., once weekly) to alleviate, prevent the onset of, or slow the development of autoimmune, alloimmune, inflammatory, and/or mitochondrial conditions. In some embodiments, methods and compositions described herein are useful for alleviating, slowing, or preventing the onset of autoimmune, alloimmune, and inflammatory conditions associated with atopy, perianal fistulas, Canine Pemphigus foliaceus, Sebaceous adenitis, Granulomatous Meningoencephalitis, Inflammatory Bowel Disease, or stomatitis.

### BACKGROUND

Calcineurin inhibitors are used as treatments for a variety of alloimmune, autoimmune, and inflammatory conditions in both human and non-human animals. They have also shown promise as treatments for conditions associated with mitochondrial dysfunction via their action on mitochondrial fluxes [Fournier, et al.]. However, their use is limited by the frequency of adverse effects associated with them, as well as the frequency with which patients are required to take their doses [Azzi, et al.].

### SUMMARY

Calcineurin inhibitors are often used as treatments for conditions related to the immune system, including, for example, cyclosporine for psoriasis [Ellis et al.] or tacrolimus to prevent rejection in liver transplantation [Haddad, et al.]. These treatments, however, are limited by the adverse effects associated with calcineurin inhibitors, as well as the frequency with which patients are required to take their doses. In cats, cyclosporine, for example, is associated with nausea and must be taken at least once daily [Roberts, et al] while tacrolimus is associated with anemia and must also be taken twice daily [Kyles, et al.]. As such, there is a need for new formulations of calcineurin inhibitors which could alleviate these undesirable conditions.

Calcineurin inhibitors are metabolized via cytochrome p450 enzymes. Without being bound by theory, the present disclosure encompasses an insight that inhibitors of cytochrome p450, when administered simultaneously with a calcineurin inhibitor, can result in a longer half-life of the calcineurin inhibitor in the body as well as a slower rate of decline of levels of the calcineurin inhibitor in the bloodstream [Dresser et al.].

In one aspect, the present disclosure provides a method for alleviating autoimmune, alloimmune, inflammatory conditions, and mitochondrial conditions, the method comprising administering a calcineurin inhibitor, or a pharmaceutically acceptable salt thereof, and a cytochrome p450 inhibitor, or a pharmaceutically acceptable salt thereof, to a subject or biological sample. In some embodiments, the autoimmune, alloimmune, or inflammatory condition is associated with an elevated level of lymphokines or interleukins. In some embodiments, the mitochondrial condition is associated with the mitochondrial permeability transition pore (MPTP).

In another aspect, the disclosure provides a method of preventing adverse effects associated with the metabolism of calcineurin inhibitors, the method comprising contacting calcineurin with a calcineurin inhibitor, or a pharmaceutically acceptable salt thereof; and contacting cytochrome p450 with a cytochrome p450 inhibitor, or a pharmaceutically acceptable salt thereof.

In some embodiments, a calcineurin inhibitor is selected from the group consisting of cyclosporine, tacrolimus, and pimecrolimus and analogs or derivatives thereof. In some embodiments, a calcineurin inhibitor is cyclosporine.

In some embodiments, a calcineurin inhibitor is administered orally at a dose of about 0.2 mg/kg of body weight per week.

In some embodiments, a cytochrome p450 inhibitor is amiodarone, chloroquine, cimetidine, clomipramine, diphenhydramine, fluoxetine, fluphenazine, haloperidol, paroxetine, perphenazine, propafenone, propoxyphene, quinacrine, quinidine, sertraline, terbinafine, thioridazine, amiodarone, amprenavir, clarithromycin, danazol, delavirdine, diltiazem, efavirenz, erythromycin, ethinylestradiol, fluconazole, fluvoxamine, grapefruit juice, indinavir, itraconazole, ketoconazole, nefazodone, nelfinavir, quinine, ritonavir, saquinavir, Synercid, troleandomycin, verapamil, or zafirlukast. In some embodiments, a cytochrome p450 inhibitor is ritonavir.

In some embodiments, a cytochrome p450 inhibitor is administered at a dose of about 1 mg/kg/week

In certain aspects, the present disclosure provides a method of treating autoimmune, alloimmune, inflammatory, or mitochondrial conditions, the method comprising identifying a subject experiencing one or more of those conditions; administering to said subject a calcineurin inhibitor, or a pharmaceutically acceptable salt thereof; and a cytochrome p450 inhibitor, or a pharmaceutically acceptable salt thereof; one time per week.

### DETAILED DESCRIPTION

### Calcineurin Inhibitors

Calcineurin inhibitors are widely used for autoimmune, alloimmune, and inflammatory conditions. Cyclosporine, for example, is used to prevent organ transplant rejection, an alloimmune condition, as well as to treat chronic idiopathic urticaria, an inflammatory condition. Without being bound by theory, it is understood that the mechanism by which calcineurin inhibitors are able to treat these conditions is to bind to the cytosolic protein cyclophin in lymphocytes, and thereby inhibit calcineurin in the calcineurin-phosphatase pathway. This lowers the activity of T cells, an important type of white blood cell. This can also have downregulating effects on the immune system at large [Reynolds et al.].

Calcineurin inhibitors have also been used to treat conditions associated with mitochondrial dysfunction. Cyclosporine, for example, has been used to treat muscular dystrophy, a condition associated with mitochondrial dysfunction [Hicks et al.]. Without being bound by theory, it is understood that the mechanism by which calcineurin inhibitors treat mitochondrial conditions is by inhibiting the MPTP, increasing the survivability of the mitochondria [Halestrap et al.].

Unfortunately, high levels of calcineurin inhibitors can also lead to adverse effects. For example, it was found that cyclosporine blood concentration of greater than 250 ng/mL can lead to adverse effects over the long term in severe ulcerative colitis, including hypertension and nephrotoxicity [Pham et al.]. This can be difficult to maintain, however, as the blood concentration of cyclosporine spikes in the first 2 hours after dosing and then drops rapidly in 4 hours after dosing [Gomez et al.]. Similar results are found with other calcineurin inhibitors.

Previously, it has been found that if a calcineurin inhibitor (e.g. cyclosporine) is given sometime after a cytochrome p450 inhibitor (e.g. itraconazole), the dosing schedule of cyclosporine may be extended up to 4 hours. However, it was assumed the only way to extend the dosing schedule of cyclosporine to a once daily schedule is by co-administering a calcineurin inhibitor and a cytochrome p450 inhibitor with a cola [Wimberley et al.].

Also, previously, it had been assumed that the only way to improve the pharmacokinetic linearity of a calcineurin inhibitor was by changing the formulation of the calcineurin inhibitor, such as the microemulsion formulation of cyclosporine known as Neoral [Mueller et al.].

The present disclosure encompasses an insight that cytochrome p450 inhibitors, if given simultaneously or near simultaneously in combination with calcineurin inhibitors, can allow for a more sustained concentration of calcineurin in the bloodstream with a less dramatic drop in concentration over the first 4 hours of dosing. Calcineurin inhibitors are metabolized by cytochrome p450, and inhibition of cytochrome p450 in combination with an active drug leads to a longer half-life of the drug.

The present disclosure also encompasses an insight that administering a cytochrome p450 inhibitor simultaneously or near simultaneously in combination with a calcineurin inhibitor would allow for dosing frequency to improve patient compliance, e.g., once weekly, twice weekly, and the like.

The presently claimed methods and compositions alleviate a variety of inflammatory, alloimmune, autoimmune, and/or mitochondrial conditions.

The present disclosure encompasses an insight that a combination of inhibition of calcineurin and inhibition of cytochrome p450 can alleviate inflammation, alloimmunity, and autoimmunity, associated with, in some embodiments, high levels of lymphocytes. It also encompasses an insight that a combination of inhibition of calcineurin and inhibition of cytochrome p450 can alleviate mitochondrial dysfunction.

### Compositions

In certain aspects, methods described herein include the manufacture and use of pharmaceutical compositions and medicaments that include compounds identified by a method described herein as active ingredients. Also included are the pharmaceutical compositions themselves.

In one or more embodiments, a calcineurin inhibitor is selected from the group consisting of cyclosporine, tacrolimus, and pimecrolimus and analogs or derivatives thereof. In some embodiments, a calcineurin inhibitor is cyclosporine.

In one or more embodiments, a cytochrome p450 inhibitor is amiodarone, chloroquine, cimetidine, clomipramine, diphenhydramine, fluoxetine, fluphenazine, haloperidol, paroxetine, perphenazine, propafenone, propoxyphene, quinacrine, quinidine, ritonavir, sertraline, terbinafine, thioridazine, amiodarone, amprenavir, clarithromycin, danazol, delavirdine, diltiazem, efavirenz, erythromycin, ethinylestradiol, fluconazole, fluvoxamine, grapefruit juice, indinavir, itraconazole, ketoconazole, nefazodone, nelfinavir, quinine, ritonavir, saquinavir, Synercid, troleandomycin, verapamil, or zafirlukast. In some embodiments, a cytochrome p450 inhibitor is ritonavir.

In some embodiments, compositions disclosed herein include other compounds, drugs, and/or agents used for the treatment of alloimmune, autoimmune, and inflammatory conditions. For example, in some instances, compositions disclosed herein can be combined with one or more (e.g., one, two, three, four, five, or less than ten) compounds.

In some instances, compositions disclosed herein are formulated for use as or in pharmaceutical compositions. Such compositions are formulated or adapted for administration to a subject via any route, e.g., any route approved by the Food and Drug Administration (FDA). Exemplary methods are described in the FDA's CDER Data Standards Manual, version number 004 (which is available at fda.give/cder/dsm/DRG/drg00301.htm). Pharmaceutical compositions described herein can be formulated for oral, parenteral, or transdermal delivery. Compounds of the present disclosure may also be combined with other pharmaceutical agents.

In some aspects, the present disclosure provides kits that include one or more compositions comprising cyclosporine and/or ritonavir and/or itraconazole (in separate compositions or in a single composition). The kit may also include instructions for the physician and/or patient, syringes, needles, box, bottles, vials, etc.

In some instances, methods described herein comprise administration of an effective amount of a composition or compositions comprising a calcineurin inhibitor and a cytochrome p450 inhibitor (as part of a single composition, or as separate compositions), as described above. The terms “effective amount” and “effective to treat,” as used herein, refer to an amount or a concentration of one or more drugs for a period of time (including acute or chronic administration and periodic or continuous administration) that is effective within the context of its administration for causing an intended effect or physiological outcome.

In some instances, compositions comprise a calcineurin inhibitor (e.g. cyclosporine), a cytochrome p450 inhibitor (e.g. ritonavir), and a pharmaceutically acceptable carrier, adjuvant and/or vehicle. In some instances, compositions described herein further comprise one or more additional therapeutic agents in an effective amount for achieving a modulation of disease or disease symptoms.

The term “pharmaceutically acceptable carrier or adjuvant” refers to a carrier or adjuvant that may be administered to a patient, together with a compound of this present disclosure, and which does not destroy the pharmacological activity thereof and is nontoxic when administered in doses sufficient to deliver a therapeutic amount of the compound. As used herein the language “pharmaceutically acceptable carrier” includes saline, solvents, dispersion media, coatings, antibacterial and antifungal agents, isotonic and absorption delaying agents, and the like, compatible with pharmaceutical administration.

Compositions are typically formulated to be compatible with its intended route of administration. Examples of routes of administration include parenteral, e.g., intravenous, intradermal, subcutaneous, oral (e.g., inhalation), transdermal (topical), transmucosal, and rectal administration.

Compositions can be in the form of a solution or powder for inhalation and/or nasal administration. Such compositions may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

Compositions can be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase combined with emulsifying and/or suspending agents. If desired, certain sweetening and/or flavoring and/or coloring agents may be added.

Alternatively or in addition, pharmaceutical compositions can be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

In some embodiments, the present disclosure provides methods for administering a composition comprising a calcineurin inhibitor (e.g. cyclosporine) and a composition comprising a cytochrome p450 inhibitor (e.g., ritonavir), each including pharmaceutical compositions, (indicated below as ‘X’) disclosed herein in the following methods: Substance X for use as a medicament in the treatment of one or more diseases or conditions disclosed herein (e.g., inflammation, referred to in the following examples as ‘Y’). Use of substance X for the manufacture of a medicament for the treatment of Y; and substance X for use in the treatment of Y.

In some instances, therapeutic compositions disclosed herein can be formulated for sale in the US, import into the US, and/or export from the US.

The pharmaceutical compositions can be included in a container, pack, or dispenser together with instructions for administration.

### Dosage

In some embodiments, a method of treating an autoimmune, alloimmune, or inflammatory condition comprises administering a calcineurin inhibitor and a cytochrome p450 inhibitor.

In some embodiments, a calcineurin inhibitor and a cytochrome p450 inhibitor are administered contemporaneously.

In some embodiments, a calcineurin inhibitor and a cytochrome p450 inhibitor are administered sequentially.

In some aspects of the present disclosure, a calcineurin inhibitor and a cytochrome p450 inhibitor are individually administered (i.e., separate dosage forms).

In some embodiments, the calcineurin inhibitor is administered in an amount of about 0.1 mg/kg/week body weight, about 0.5 mg/kg/week body weight, about 1 mg/kg/week body weight, about 2 mg/kg/week body weight, or about 4 mg/kg/week body weight. In some embodiments, the cytochrome p450 inhibitor is administered in an amount of 1 mg/kg/week, 2 mg/kg/week, 5 mg/kg/week, or 10 mg/kg/week. In some embodiments, the cytochrome p450 inhibitor is administered in an amount of 5 mg/week, 10 mg/week, or 20 mg/week. The compounds can be administered separately or together, including as a part of a regimen of treatment.

In some aspects of the present disclosure, the calcineurin inhibitor is cyclosporine and the cytochrome p450 inhibitor is ritonavir. In some aspects, cyclosporine and ritonavir are individually administered (i.e., separate dosage forms) . . . . In some embodiments, cyclosporine is administered in amount that is about 0.1 mg/kg/week body weight, about 0.5 mg/kg/week body weight, about 1 mg/kg/week body weight, about 2 mg/kg/week body weight, or about 4 mg/kg/week body weight. In some embodiments, ritonavir is administered in an amount of 1 mg/kg/week, 2 mg/kg/week, 5 mg/kg/week, or 10 mg/kg/week. In some embodiments, the ritonavir is administered in an amount of 5 mg/week, 10 mg/week, or 20 mg/week. Compounds described herein can be administered separately or together, including as a part of a regimen of treatment.

In some embodiments, cyclosporine is administered in an amount that is about 1.0-2.5 mg/kg. In some embodiments, cyclosporine is administered in an amount that is about 1.0, 1.1, 1.2, 1.3, 1.4, 1.5, 1.6, 1.7, 1.8, 1.9, 2.0, 2.1, 2.2, 2.3, 2.4, or 2.5 mg/kg. In some embodiments, cyclosporine is administered in an amount that is about 5-15 mg. In some embodiments, cyclosporine is administered in an amount that is about 5, 6, 7, 8, 9, 10, 11, 12, 13, 14, 15, 16, 17, 18, 19, or 29 mg. In some embodiments, ritonavir is administered in an amount that is 5 mg, 10 mg, or 20 mg. The compounds can be administered separately or together, including as a part of a regimen of treatment. In some embodiments, a single dose of cyclosporine and ritonavir is administered, followed by a different dose after a period of time (e.g., one day, two days, three days, four days, five days, six days, or a week).

In some embodiments, a subject receives a specific dosage over a period of days, weeks, months, or years. For example, 1 week, 2 weeks, 3 weeks, 1 month, 2 months, 3 months, 4 months, 5 months, 6 months, 7 months, 8 months, 9 months, 10 months, 11 months, 1 year, 2 years, 3 years, 4 years, 5 years and the like. In some embodiments, a subject receives a calcineurin inhibitor and a cytochrome inhibitor according to an intermittent dosing schedule (e.g., less frequently than once per day, such as once every other day, twice per week, once per week, twice per month, once per month, twice per year, once per year, and the like). In some embodiments, a subject receives a calcineurin inhibitor and a cytochrome inhibitor once every other day. In some embodiments, a subject receives a calcineurin inhibitor and a cytochrome inhibitor twice per week. In some embodiments, a subject receives a calcineurin inhibitor and a cytochrome inhibitor once per week. In some embodiments, a subject receives a calcineurin inhibitor and a cytochrome inhibitor every other week. In some embodiments, a subject receives a calcineurin inhibitor and a cytochrome inhibitor twice per month. In some embodiments, a subject receives a calcineurin inhibitor and a cytochrome inhibitor once per month. In some embodiments, a subject receives a calcineurin inhibitor and a cytochrome inhibitor every other month. In some embodiments, a subject receives a calcineurin inhibitor and a cytochrome inhibitor twice per year. In some embodiments, a subject receives a calcineurin inhibitor and a cytochrome inhibitor once per year.

In some embodiments, a calcineurin inhibitor is cyclosporine and a cytochrome p450 inhibitor is ritonavir. In some embodiments, cyclosporine and ritonavir or itraconazole are administered, separately or together, as a single daily dosage, on a daily basis, a weekly basis or some other basis. In some embodiments, a subject receives a specific dosage over a period of weeks, months, or years. For example, 1 week, 2 weeks, 3 weeks, 1 month, 2 months, 3 months, 4 months, 5 months, 6 months, 7 months, 8 months, 9 months, 10 months, 11 months, 1 year, 2 years, 3 years, 4 years, 5 years and the like.

### Methods of Treatment

The methods described herein include methods for the treatment of disorders associated with elevated levels of lymphocytes (e.g. organ transplant rejection) or mitochondrial dysfunction (e.g. muscular dystrophy). Generally, the methods include administering a therapeutically effective amount of a calcineurin inhibitor (e.g., cyclosporine) in combination with a cytochrome p450 inhibitor (e.g. itraconazole) as described herein, to a subject (e.g., a mammalian subject, e.g., a human subject) who is in need of, or who has been determined to be in need of, such treatment.

In some instances, methods can include selection of a human subject who has or had a condition or disease. In some instances, suitable subjects include, for example, subjects who have or had a condition or disease but that resolved the disease or an aspect thereof, present reduced symptoms of disease (e.g., relative to other subjects (e.g., the majority of subjects) with the same condition or disease), and/or that survive for extended periods of time with the condition or disease (e.g., relative to other subjects (e.g., the majority of subjects) with the same condition or disease), e.g., in an asymptomatic state (e.g., relative to other subjects (e.g., the majority of subjects) with the same condition or disease).

The methods disclosed herein can be applied to a wide range of species, e.g., humans, non-human primates (e.g., monkeys), horses, cattle, pigs, sheep, deer, elk, goats, dogs, cats, rabbits, guinea pigs, hamsters, rats, and mice.

The terms “treat”, “treating”, “treatment”, etc., as applied to an isolated cell, include subjecting the cell to any kind of process or condition or performing any kind of manipulation or procedure on the cell. As applied to a subject, the term “treating” refer to providing medical or surgical attention, care, or management to an individual. The individual is usually ill or injured, or at increased risk of becoming ill relative to an average member of the population and in need of such attention, care, or management.

In some embodiments, the term “treating” and “treatment” refers to administering to a subject an effective amount of a composition, e.g., a composition comprising a calcineurin inhibitor and a composition comprising a cytochrome p450 inhibitor, so that the subject has a reduction in at least one symptom of the disease or an improvement in the disease, for example, beneficial or desired clinical results. For purposes of the present disclosure, beneficial or desired clinical results include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable. Treating can refer to prolonging survival as compared to expected survival if not receiving treatment. Thus, one of skill in the art realizes that a treatment may improve the disease condition, but may not be a complete cure for the disease. In some embodiments, treatment can be prophylactic treatment, where the subject is administered a composition as disclosed herein to a subject at risk of developing inflammation as disclosed herein. In some embodiments, treatment is “effective” if the progression of a disease is reduced or halted.

The term “subject,” as used herein, refers to any animal. In some instances, the subject is a mammal. In some instances, the term “subject”, as used herein, refers to a human (e.g., a man, a woman, or a child). In some embodiments, a “subject” is a non-human mammal, e.g., non-human primates (e.g., monkeys), horses, cattle, pigs, sheep, deer, elk, goats, dogs, cats, rabbits, guinea pigs, hamsters, rats, and mice.

In some instances, subject selection can include obtaining a sample from a subject (e.g., a candidate subject) and testing the sample for an indication that the subject is suitable for selection. In some instances, the subject can be confirmed or identified, e.g. by a healthcare professional, as having had or having a condition or disease. In some instances, exhibition of a positive immune response towards a condition or disease can be made from patient records, family history, and/or detecting an indication of a positive immune response. In some instances multiple parties can be included in subject selection. For example, a first party can obtain a sample from a candidate subject and a second party can test the sample. In some instances, subjects can be selected and/or referred by a medical practitioner (e.g., a general practitioner). In some instances, subject selection can include obtaining a sample from a selected subject and storing the sample and/or using the methods disclosed herein. Samples can include, for example, cells or populations of cells.

In some instances, treatment methods can include a single administration, multiple administrations, and repeating administration as required for the prophylaxis or treatment of the disease or condition from which the subject is suffering. In some instances treatment methods can include assessing a level of disease in the subject prior to treatment, during treatment, and/or after treatment. In some instances, treatment can continue until a decrease in the level of disease in the subject is detected.

The terms “administer,” “administering,” or “administration,” as used herein refers to implanting, absorbing, ingesting, injecting, or inhaling, the inventive drug, regardless of form. In some instances, one or more of the compounds disclosed herein can be administered to a subject topically (e.g., nasally) and/or orally. For example, the methods herein include administration of an effective amount of compound or compound composition to achieve the desired or stated effect. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Following administration, the subject can be evaluated to detect, assess, or determine their level of disease. In some instances, treatment can continue until a change (e.g., reduction) in the level of disease in the subject is detected.

Upon improvement of a patient's condition (e.g., a change (e.g., decrease) in the level of disease in the subject), a maintenance dose of a compound, composition or combination of this present disclosure may be administered, if necessary. Subsequently, the dosage or frequency of administration, or both, may be reduced, as a function of the symptoms, to a level at which the improved condition is retained. Patients may, however, require intermittent treatment on a long-term basis upon any recurrence of disease symptoms.

### Definitions

Neurodegeneration refers to any condition that results in the progressive death of nerve cells.

Mitochondrial condition refers to any conditions that results from the dysfunction of mitochondria.

Inhibitory agent: As used herein, the term “inhibitory agent” refers to an entity, condition, or event whose presence, level, or degree correlates with decreased level or activity of a target). In some embodiments, an inhibitory agent may be act directly (in which case it exerts its influence directly upon its target, for example by binding to the target); in some embodiments, an inhibitory agent may act indirectly (in which case it exerts its influence by interacting with and/or otherwise altering a regulator of the target, so that level and/or activity of the target is reduced). In some embodiments, an inhibitory agent is one whose presence or level correlates with a target level or activity that is reduced relative to a particular reference level or activity (e.g., that observed under appropriate reference conditions, such as presence of a known inhibitory agent, or absence of the inhibitory agent in question, etc).

An inhibitor, as used herein, refers to an inhibitory agent, while inhibition refers to the activity of an inhibitor agent.

Regulating: regulating refers to altering, enhancing, or diminishing the activities or an organelle or cell.

Antagonist: Those skilled in the art will appreciate that the term “antagonist”, as used herein, may be used to refer to an agent, condition, or event whose presence, level, degree, type, or form correlates with decreased level or activity of another agent (i.e., the inhibited agent, or target). In general, an antagonist may be or include an agent of any chemical class including, for example, small molecules, polypeptides, nucleic acids, carbohydrates, lipids, metals, and/or any other entity that shows the relevant inhibitory activity. In some embodiments, an antagonist may be direct (in which case it exerts its influence directly upon its target); in some embodiments, an antagonist may be indirect (in which case it exerts its influence by other than binding to its target; e.g., by interacting with a regulator of the target, so that level or activity of the target is altered).

Agonist: Those skilled in the art will appreciate that the term “agonist” may be used to refer to an agent, condition, or event whose presence, level, degree, type, or form correlates with increased level or activity of another agent (i.e., the agonized agent or the target agent). In general, an agonist may be or include an agent of any chemical class including, for example, small molecules, polypeptides, nucleic acids, carbohydrates, lipids, metals, and/or any other entity that shows the relevant activating activity. In some embodiments, an agonist may be direct (in which case it exerts its influence directly upon its target); in some embodiments, an agonist may be indirect (in which case it exerts its influence by other than binding to its target; e.g., by interacting with a regulator of the target, so that level or activity of the target is altered).

Administration: As used herein, the term “administration,” typically refers to application or delivery to a subject or system. Those of ordinary skill in the art, reading the present disclosure, will appreciate, for example, that a variety of routes are available for administration of compositions; for example, some compositions may be administered by one or more routes such as ocular, oral, parenteral, topical, etc., In some particular embodiments, administration may be bronchial (e.g., by bronchial instillation), buccal, dermal (which may be or comprise, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e. g. intrahepatic), mucosal, nasal, oral, rectal, subcutaneous, sublingual, topical, tracheal (e.g., by intratracheal instillation), vaginal, vitreal, etc. Furthermore, the present disclosure, in some embodiments, describes administration of behavioral therapy, for example via interaction with a counselor (e.g., a therapist) and/or with a device or computing system as described herein. In some embodiments, administration may involve dosing, application, or interaction that is intermittent (e.g., a plurality of doses separated in time, for example, in some embodiments, less frequently than once per day, such as once every other day, twice per week, once per week, twice per month, once per month, twice per year, once per year, and the like). In some embodiments, administration of a calcineurin inhibitor and a cytochrome p450 inhibitor to the subject is according to an intermittent dosing schedule. In some embodiments, administration of a calcineurin inhibitor and a cytochrome p450 inhibitor to the subject occurs less frequently than once per day. In some embodiments, a calcineurin inhibitor and a cytochrome p450 inhibitor are each administered to the subject is once per week. In some embodiments, a calcineurin inhibitor and a cytochrome p450 inhibitor are each administered to the subject is twice per week. In some embodiments, a calcineurin inhibitor and a cytochrome p450 inhibitor are each administered to the subject is once per month. In some embodiments, a calcineurin inhibitor and a cytochrome p450 inhibitor are each administered to the subject is twice per month. In some embodiments, a calcineurin inhibitor and a cytochrome p450 inhibitor are each administered to the subject is twice per year. In some embodiments, a calcineurin inhibitor and a cytochrome p450 inhibitor are each administered to the subject is once per year.

Cyclosporine is a chemical compound with the following structure

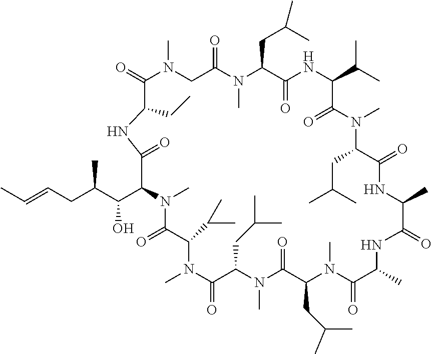 [SMILES: C/C=C/C[C@@H](C)[C@@H](O)[C@H]1C(=O)N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

Ritonavir is a chemical compound with the following structure:

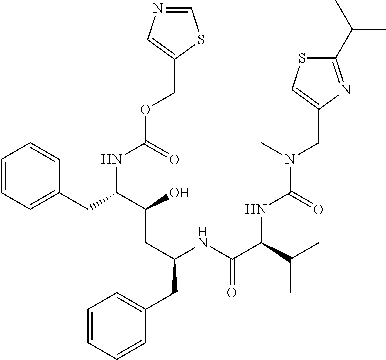 [SMILES: CC(C)c1nc(CN(C)C(=O)N[C@H](C(=O)N[C@@H](Cc2ccccc2)C[C@H](O)[C@H](Cc2ccccc2)NC(=O)OCc2cncs2)C(C)C)cs1]

About: The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Pharmaceutically Acceptable Salts: The term “pharmaceutically acceptable salt” or “pharmaceutically acceptable salts” refers to a salt formed from an acid and a basic group of a pharmaceutically active compounds. Illustrative salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts.

Unless otherwise defined, all technical and scientific terms used herein have the same meaning as commonly understood by one of ordinary skill in the art to which this present disclosure belongs. Methods and materials are described herein for use in the present present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the present disclosure will be apparent from the following detailed description and figures, and from the claims.

The present disclosure is further described in the following examples, which do not limit the scope of the present disclosure described in the claims.

### EXEMPLIFICATION

The present disclosure is further described in the following examples, which do not limit the scope of the present disclosure described in the claims.

### Example 1

### Materials and Methods

Compounds are tested in a rising dose study as described herein.

Modified cyclosporine (Neoral) and ritonavir are obtained.

18 cats are screened for inclusion to the rising dose study. Inclusion criteria are greater than 6 months of age and a bodyweight of 3.5-6 kg. Exclusion criteria are the evidence of any clinically significant (in the opinion of the Investigator) acute or chronic disease following a detailed medical and surgical history and a complete physical examination.

Following admittance, subjects will be divided into 3 cohorts: cohort 1, cohort 2, and cohort 3. Each subject will participate in only one cohort. Each cohort will contain 6 cats.

Cohort 1 will receive an oral dose of 1.2 mg cyclosporine coadministered with a single oral dose of 5 mg ritonavir.

Cohort 2 will receive an oral dose of 1.2 mg cyclosporine coadministered with a single oral dose of 10 mg ritonavir.

Cohort 3 will receive an oral dose of 1.2 mg cyclosporine coadministered with a single oral dose of 20 mg ritonavir.

Each cohort will be sampled for 7 days following oral dose of cyclosporine and oral dose of coadministered cyclosporine and ritonavir.

### Results

Whole blood cyclosporine concentrations will be determined for each sample. Also, for each sample, whole blood PK parameters will be estimated using noncompartmental analysis, as appropriate: Cmax, tmax, kel, t½, AUC0-last, AUC0-inf, CL/F, and Vz/F. Also, plasma ritonavir concentrations will be measured to confirm its presence after Day 4 dosing.

Descriptive statistics for all relevant PK parameters will be calculated: n, mean, standard deviation, minimum, median, maximum, geometric mean, and coefficient of variation.

PK parameters—Cmax, AUC0-last, AUC0-inf—will be compared between Day 1 and Day 4 using an analysis of variance (ANOVA) model with subject as a random effect and day as a fixed effect, using the natural logarithms of the parameters uncorrected for dose. Confidence intervals (CI) (90%) will be constructed for the geometric mean ratios (GMR) of cyclosporine on Day 4 to Day 1 for all three parameters using the log transformed data and the two one-sided t-tests procedure. The GMRs and 90% CIs will be exponentiated back to the original scale. The effects of coadministration of ritonavir on cyclosporine will be evaluated from the GMRs and CIs.

This analysis will show a statistically significant effect for the prolongation of the cyclosporine half-life appropriate for a once-weekly dosing regime.

### Example 2

### Materials and Methods

Compounds were tested in a comparative dose study as described herein.

Modified cyclosporine (Atopica) and ritonavir were obtained.

14 cats were screened for inclusion to the rising dose study. Of those, 12 were selected. Inclusion criteria were greater than 6 months of age and a bodyweight of 3.5-6 kg. Exclusion criteria were the evidence of any clinically significant (in the opinion of the Investigator) acute or chronic disease following a detailed medical and surgical history and a complete physical examination, as well as any other factors, such as recent medical treatment, that would impact the study objectives according to the opinion of the Investigator.

Following admittance, subjects were acclimated to study conditions for 7 days prior to the start of the study. The subjects received 10 mg of modified cyclosporine in a Torpac capsule and their conditions was followed for 7 days.

Physical measurements and clinical observations were performed throughout the study. Also, blood samples were taken once before the 7 day period following dosing, once after the study period, and 15 times throughout the 7 day period. Blood samples were analyzed.

Following a 10 day washout, period 2 began. Subjects received 10 mg of cyclosporine (Atopica) in a Torpac capsule and 20 mg of ritonavir in solution in a Torpac capsule. An identical procedure was followed with regards to physical measurements, clinical observations, and blood samples.

### Results

Whole blood cyclosporine concentrations were determined for each sample. Also, for each sample, whole blood PK parameters were estimated using noncompartmental analysis, as appropriate: Cmax, tmax, kel, t½, AUC0-last, AUC0-inf, CL/F, and Vz/F. Also, plasma ritonavir concentrations were measured for period 2:11 time points within the first 24 hours.

Descriptive statistics for all relevant PK parameters will be calculated: n, mean, standard deviation, minimum, median, maximum, geometric mean, and coefficient of variation.

PK parameters—Cmax, AUC0-last, AUC0-inf—will be compared between Day 1 and Day 4 using an analysis of variance (ANOVA) model with subject as a random effect and day.

The table below provides a summary of pharmacokinetic parameters for cyclosporine after oral administration of 10 mg with and without ritonavir (20 mg) to cats. Two cats were excluded from the study due to dosing variability.

| | Cyclosporine | | Cyclosporine | |
|---|---|---|---|---|
| Parameter* | Alone | | With Ritonavir | |
| Cmax (ng/mL) | 495 | [95.2] (9) | 977 | [042] (9) |
| Tmax (hr) | 1.50 (9) | [1.00-4.00] | 2.00 (9) | [1.00-6.00] |
| AUC(0-t) (hr × ng/mL) | 3,463 | [82.2] (9) | 15,556 | [081] (9) |
| AUC(inf) (hr × ng/mL) | 3,848 | [87.4] (8) | 19,924 | [035] (8) |
| λz (1/hr) | 0.0280 | [119] (8) | 0.0121 | [24.3] (8) |
| t½ (hr) | 24.7 | [119] (8) | 57.4 | [243] (8) |

*Geometric mean [geometric CV %] (N) except Tmax for which median (N) [Range] is reported.

### REFERENCES

1. Fournier, N., G. Ducet, and A. Crevat. “Action of cyclosporine on mitochondrial calcium fluxes.” Journal of bioenergetics and biomembranes 19.3 (1987): 297-303.
2. Jamil R. Azzi, Mohamed H. Sayegh, Samir G. Mallat, Calcineurin Inhibitors: 40 Years Later, Can't Live Without . . . , The Journal of Immunology Dec. 15, 2013, 191 (12) 5785-5791; DOI: 10.4049/jimmunol.1390055
3. Jeffrey Schiff, Edward Cole and Marcelo Cantarovich, Therapeutic Monitoring of Calcineurin Inhibitors for the Nephrologist, CJASN March 2007, 2 (2) 374-384; DOI: https://doi.org/10.2215/CJN.03791106
4. van Hooff, Johannes P.; Christiaans, Maarten H. L.; van Duijnhoven, Elly M. Tacrolimus and Posttransplant Diabetes Mellitus in Renal Transplantation, Transplantation: Jun. 15, 2005-Volume 79-Issue 11-p 1465-1469 doi: 10.1097/01.TP.0000157870.21957.E5
5. Ellis C N, Gorsulowsky D C, Hamilton T A, et al. Cyclosporine Improves Psoriasis in a Double-blind Study. JAMA. 1986; 256 (22): 3110-3116. doi: 10.1001/jama. 1986.03380220076026
6. Haddad E, McAlister V, Renouf E, Malthaner R, Kjaer M S, Gluud L L. Cyclosporin versus tacrolimus for liver transplanted patients. Cochrane Database of Systematic Reviews 2006, Issue
4. Art. No.: CD005161. DOI: 10.1002/14651858.CD005161.pub2. Accessed 20 May 2021.
7. Niwa, Toshiro, et al. “Effect of cyclosporine and tacrolimus on cytochrome p450 activities in human liver microsomes.” Yakugaku Zasshi 127.1, 2007:209-216.
8. Dresser, G. K., Spence, J. D. & Bailey, D. G. Pharmacokinetic-Pharmacodynamic Consequences and Clinical Relevance of Cytochrome P450 3A4 Inhibition. Clin Pharmacokinet 38, 41-57, 2000. https://doi.org/10.2165/00003088-200038010-00003
9. Reynolds, N. J. and Al-Daraji, W. I., Calcineurin inhibitors and sirolimus: mechanisms of action and applications in dermatology. Clinical and Experimental Dermatology, (2002), 27:555-561. https://doi.org/10.1046/j.1365-2230.2002.01148.x
10. Hicks, Debbie, et al. “Cyclosporine A treatment for Ullrich congenital muscular dystrophy: a cellular study of mitochondrial dysfunction and its rescue.” Brain 132.1 (2009): 147-155.
11. Halestrap, A P et al. “Cyclosporin A binding to mitochondrial cyclophilin inhibits the permeability transition pore and protects hearts from ischaemia/reperfusion injury.” Molecular and cellular biochemistry vol. 174, 1-2 (1997): 167-72.
12. Pham C Q, Efros C B, Berardi R R. Cyclosporine for Severe Ulcerative Colitis. Annals of Pharmacotherapy. 2006; 40 (1): 96-101. doi: 10.1345/aph.1G374
13. Gomez, D. Y., Wacher, V. J., Tomlanovich, S. J., Hebert, M. F. and Benet, L. Z., The effects of ketoconazole on the intestinal metabolism and bioavailability of cyclosporine. Clinical Pharmacology & Therapeutics, 1995, 58:15-19. https://doi.org/10.1016/0009-9236 (95) 90067-5
14. Wimberley, S., Haug Iii, M., Shermock, K., Qu, A., Maurer, J., Mehta, A., Schilz, R. and Gordon, S. (2001), Enhanced cyclosporine-itraconazole interaction with cola in lung transplant recipients. Clin Transplantation, 15:116-122. doi.org/10.1034/j.1399-0012.2001.150206.x
15. Mueller, E. A., Kovarik, J. M., van Bree, J. B. et al. Improved Dose Linearity of Cyclosporine Pharmacokinetics from a Microemulsion Formulation. Pharm Res 11, 301-304 (1994). doi.org/10.1023/A: 1018923912135.
16. Roberts, et al., Safety, tolerability, and pharmacokinetics of 6-month daily dosing of an oral formulation of cyclosporine (ATOPICA for Cats®) in cats, J. Veterinary Pharmacology and Therapeutics, 37 (2): 161-168 (2013).
17. Kyles, et al., Pharmacokinetics of tacrolimus after multidose oral administration and efficacy in the prevention of allograft rejection in cats with renal transplants, Am. J. of Veterinary Research, 64 (7): 926-934 (2003).

1. A method of treating an alloimmune, autoimmune, inflammatory, or mitochondrial condition in a subject, the method comprising: administering a calcineurin inhibitor and a cytochrome p450 inhibitor to the subject according to an intermittent dosing schedule.

2. The method of claim 1, wherein the calcineurin inhibitor and the cytochrome p450 inhibitor are administered once weekly.

3. The method of claim 1, wherein the calcineurin inhibitor and the cytochrome p450 inhibitor are administered contemporaneously.

4. The method of claim 1, wherein the calcineurin inhibitor and the cytochrome p450 inhibitor are administered sequentially.

5. The method of any one of claims 1-3, wherein the calcineurin inhibitor is selected from: cyclosporine, tacrolimus, pimecrolimus, and analogs or derivatives thereof, or a pharmaceutically acceptable salt thereof.

6. The method of any one of claims 1-4, wherein the cytochrome p450 inhibitor is selected from: amiodarone, chloroquine, cimetidine, clomipramine, diphenhydramine, fluoxetine, fluphenazine, haloperidol, paroxetine, perphenazine, propafenone, propoxyphene, quinacrine, quinidine, ritonavir, sertraline, terbinafine, thioridazine, amiodarone, amprenavir, clarithromycin, danazol, delavirdine, diltiazem, efavirenz, erythromycin, ethinylestradiol, fluconazole, fluvoxamine, grapefruit juice, indinavir, itraconazole, ketoconazole, nefazodone, nelfinavir, quinine, ritonavir, saquinavir, Synercid, troleandomycin, verapamil, zafirlukast, and analogs or derivatives thereof, or a pharmaceutically acceptable salt thereof.

7. The method of claim 1, wherein the calcineurin inhibitor is cyclosporine.

8. The method of claim 1, wherein the cytochrome p450 inhibitor is ritonavir.

9. The method of any one of claims 1-7, wherein the calcineurin inhibitor is administered in an amount that is about 0.1 mg/kg body weight per week, about 0.2 mg/kg body weight per week, about 0.3 mg/kg body weight per week, about 0.4 mg/kg body weight per week, about 0.5 mg/kg body weight per week, about 1.0 mg/kg body weight per week, about 2.0 mg/kg body weight per week, or about 4.0 mg/kg body weight per week.

10. The method of any one of claims 1-8, wherein the cytochrome p450 inhibitor is administered in an amount that is about 0.1 mg/kg body weight per week, about 0.2 mg/kg body weight per week, about 0.3 mg/kg body weight per week, about 0.4 mg/kg body weight per week, about 0.5 mg/kg body weight per week, about 1.0 mg/kg body weight per week, about 2.0 mg/kg body weight per week, or about 4.0 mg/kg body weight per week.

11. The method of any one of claims 1-8, wherein the cytochrome p450 inhibitor is administered in an amount that is about 1 mg per week, about 2 mg week, about 5 mg per week, about 10 mg per week, about 20 mg per week, or about 40 mg per week.

12. The method of any one of claims 1-11, wherein the autoimmune, alloimmune, or inflammatory condition is associated with elevated levels of lymphocytes.

13. The method of any one of claims 1-11, wherein the mitochondrial condition is associated with the mitochondrial permeability transition pore.

14. The method of claim 12, wherein the autoimmune, alloimmune, or inflammatory condition is atopy, perianal fistulas, Canine Pemphigus foliaceus, Sebaceous adenitis, Granulomatous Meningoencephalitis, Inflammatory Bowel Disease, or stomatitis.

15. The method of any one of claims 1-14, wherein the calcineurin inhibitor is administered as an oral dosage form.

16. The method of any one of claims 1-15, wherein the cytochrome p450 inhibitor is administered as an oral dosage form.

17. A method of mediating a disease, disorder, or condition associated with elevated levels of lymphocytes in a subject, the method comprising administering a calcineurin inhibitor and a cytochrome p450 inhibitor to the subject according to an intermittent dosing schedule.

18. A method of mediating a disease, disorder, or condition associated with the mitochondrial permeability transition pore in a subject, the method comprising administering a calcineurin inhibitor and a cytochrome p450 inhibitor to the subject according to an intermittent dosing schedule.

19. The method of claim 17 or 18, wherein the calcineurin inhibitor and the cytochrome p450 inhibitor are administered contemporaneously.

20. The method of any one of claims 17-19, wherein the calcineurin inhibitor and the cytochrome p450 inhibitor are administered sequentially.

21. The method of any one of claims 17-20, wherein the calcineurin inhibitor is selected from: cyclosporine, tacrolimus, and pimecrolimus and analogs or derivatives thereof, or a pharmaceutically acceptable salt thereof.

22. The method of any one of claims 17-21, wherein the cytochrome p450 inhibitor is selected from: amiodarone, chloroquine, cimetidine, clomipramine, diphenhydramine, fluoxetine, fluphenazine, haloperidol, paroxetine, perphenazine, propafenone, propoxyphene, quinacrine, quinidine, ritonavir, sertraline, terbinafine, thioridazine, amiodarone, amprenavir, clarithromycin, danazol, delavirdine, diltiazem, efavirenz, erythromycin, ethinylestradiol, fluconazole, fluvoxamine, grapefruit juice, indinavir, itraconazole, ketoconazole, nefazodone, nelfinavir, quinine, ritonavir, saquinavir, Synercid, troleandomycin, verapamil, or zafirlukast and analogs or derivatives thereof, or a pharmaceutically acceptable salt thereof.

23. The method of any one of claims 17-22, wherein the calcineurin inhibitor and the cytochrome p450 inhibitor are administered once weekly.

24. The method of any one of claims 1-23, wherein the subject is a non-human mammal.

25. The method of claim 24, wherein the subject is a cat or a dog.
